(19) United States
(12) Patent Application Publication
(10) Pub. No.: US 2023/0057704 A1
(43) Pub. Date: Feb. 23, 2023

(54) DISINFECTION DEVICE AND DISINFECTION SYSTEM

(71) Applicant: TOYOTA JIDOSHA KABUSHIKI KAISHA, Toyota-shi Aichi-ken (JP)

(72) Inventors: Ryota Tomizawa, Susono-shi Shizuoka-ken (JP); Shozo Takaba, Chiryu-shi Aichi-ken (JP); Ayako Shimizu, Numazu-shi Shizuoka-ken (JP); Hojung Jung, Sunto-gun Shizuoka-ken (JP); Daisuke Sato, Toyota-shi Aichi-ken (JP); Yasuhiro Kobatake, Nagoya-shi Aichi-ken (JP)

(73) Assignee: TOYOTA JIDOSHA KABUSHIKI KAISHA, Toyota-shi Aichi-ken (JP)

(21) Appl. No.: 17/844,799

(22) Filed: Jun. 21, 2022

(30) Foreign Application Priority Data
Aug. 19, 2021 (JP) 2021-134407

(51) Int. Cl. A61L 2/18; E04F 11/18

(52) U.S. Cl. CPC A61L 2/18; E04F 11/18; A61L 2209/111

(57) ABSTRACT

A disinfection device is attached to a body support that is gripped by a hand, and disinfects the body support. The disinfection device includes a main body portion that moves along the body support while in contact with the body support. The main body portion includes a wiping member configured to be impregnated with a disinfectant solution at a part where the wiping member contacts with the body support.

### CROSS-REFERENCE TO RELATED APPLICATION

This application claims priority to Japanese Patent Application No. 2021-134407 filed on Aug. 19, 2021, incorporated herein by reference in its entirety.

### BACKGROUND

### 1. Technical Field

The present disclosure relates to a disinfection device and a disinfection system.

### 2. Description of Related Art

Japanese Unexamined Patent Application Publication No. 2005-213959 discloses, as a handrail disclosed in the related art, a handrail filled with a deodorant in order to add a deodorizing function and an air purifying function to the handrail.

### SUMMARY

In various places such as buildings and vehicles, a body support that can be gripped by a hand, such as a handrail or a hanging strap, is provided for the purpose of supporting a body or assisting walking. Since such a body support is used by an unspecified number of people, it is useful to disinfect the gripped portion (surface) thereof. However, the handrail disclosed in the related art deodorizes and purifies the air around the handrail, and does not disinfect the gripped portion of the handrail.

The present disclosure provides a disinfection device and a disinfection system that disinfect a gripped portion of a body support.

A disinfection device according to a first aspect of the disclosure is attached to a body support that is gripped by a hand, and disinfects the body support. The disinfection device includes a main body portion configured to move along the body support while in contact with the body support. The main body portion includes a wiping member configured to be impregnated with a disinfectant solution at a part where the wiping member contacts with the body support.

A disinfection system according to a second aspect of the disclosure includes a disinfection device which is attached to a body support that is gripped by a hand, and disinfects automatically the body support, a sensor configured to recognize surrounding situations of the disinfection device, and a control device configured to remotely control the disinfection device based on the surrounding situations of the disinfection device recognized by the sensor.

With each aspect of the present disclosure, the gripped portion of the body support can be disinfected.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a plan view of a disinfection device according to an embodiment of the present disclosure;

FIG. 2 is a front view of the disinfection device as viewed from direction A of FIG. 1;

FIG. 3 is a cross-sectional view of the disinfection device along line of FIG. 1;

FIG. 4 is a cross-sectional view of the disinfection device along line IV-IV of FIG. 2;

FIG. 5 is a schematic configuration diagram of a disinfection system according to an embodiment of the present disclosure, which is formed in a bus;

FIG. 6 is a flowchart illustrating an example of a process performed by a control device to control operations of each disinfection device attached to a hanging strap in the bus; and

FIG. 7 is a cross-sectional view of a disinfection device according to a modification example of the present disclosure.

### DETAILED DESCRIPTION OF EMBODIMENTS

Hereinafter, each embodiment of the present disclosure will be described in detail with reference to the drawings. In the following description, similar components are given the same reference numerals and letters. In the present specification, the term “disinfection” includes not only detoxifying bacteria and viruses but also reducing bacteria and viruses (sterilization).

### First Embodiment

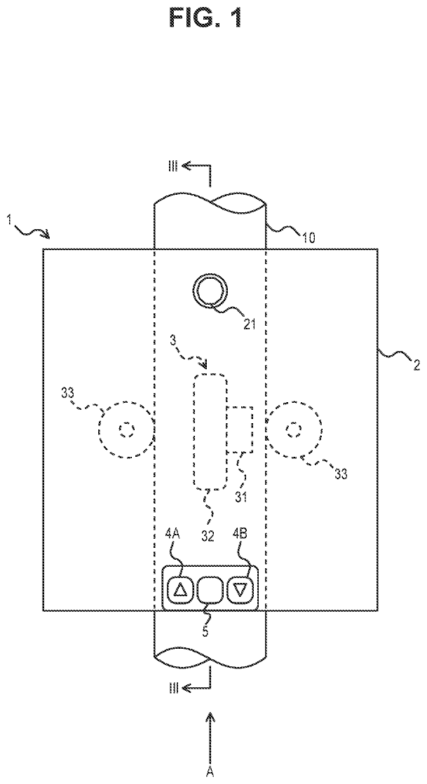

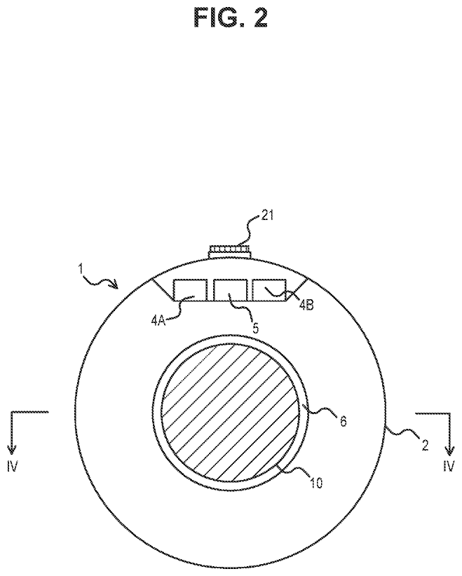

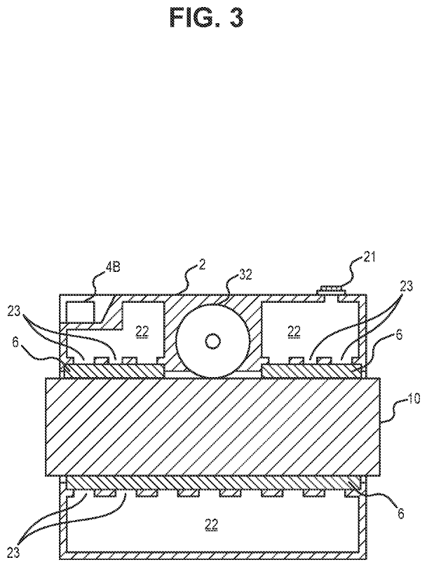

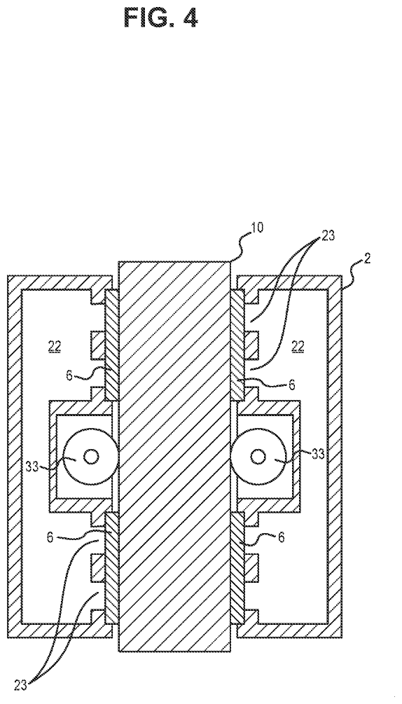

FIGS. 1 to 4 are each schematic configuration diagrams of a disinfection device 1 according to a first embodiment of the present disclosure which is attached to a body support 10 such as a handrail or a hanging strap that can be gripped by a hand and is used for disinfecting a gripped portion (surface) of the body support 10. Specifically, FIG. 1 is a plan view of the disinfection device 1, and FIG. 2 is a front view of the disinfection device 1 as viewed from direction A of FIG. 1. FIG. 3 is a cross-sectional view of the disinfection device 1 along line III-III of FIG. 1, and FIG. 4 is a cross-sectional view of the disinfection device 1 along line IV-IV of FIG. 2. In addition, in FIGS. 1 to 4, as the body support 10, a rod-shaped handrail having a circular cross section is shown.

As illustrated in FIGS. 1 to 4, the disinfection device 1 according to the present embodiment includes a device main body 2, a drive unit 3 provided inside the device main body 2, device drive buttons 4A and 4B, and a lighting lamp 5.

The device main body 2 is a hollow cylindrical member formed so as to be insertable into the body support 10. The device main body 2 is inserted through the body support 10 such that its inner peripheral surface is in contact with a gripped portion of the body support 10. The device main body 2 is attached to the body support 10 such that its inner peripheral surface can be reciprocated along the axial direction of the body support 10 while being in contact with the gripped portion of the body support 10.

Inside the device main body 2, a space (hereinafter, referred to as a “storage portion”) 22 for storing a disinfectant solution replenished from a disinfectant liquid replenishment hole 21 formed on the outer peripheral side of the device main body 2 is formed. A wiping member 6 having liquid absorption retention is attached to the inner peripheral surface of the device main body 2 over the entire circumference in the circumferential direction. The storage portion 22 and the wiping member 6 communicate with each other through a communication hole 23 formed on the inner peripheral side of the device main body 2. As a result, the disinfectant solution stored in the storage portion 22 can be impregnated into the wiping member.

The drive unit 3 is a component for moving the device main body 2 along the axial direction of the body support 10. The drive unit 3 includes a motor 31, a drive wheel 32 driven by the motor 31, and two auxiliary wheels 33.

The motor 31 has a built-in motor driver and battery (not illustrated), and in the present embodiment, the motor 31 is driven when any one of the device drive buttons 4A and 4B is pressed. In this case, when the device drive button 4A is pressed, the motor 31 is controlled such that the rotation direction of the motor 31 is opposite to that when the device drive button 4B is pressed.

The drive wheel 32 is attached inside the device main body 2 such that, when the device main body 2 is attached to the body support 10, the drive wheel can be in contact with the surface of the body support 10 and roll on the surface of the body support 10 along the axial direction of the body support 10.

The auxiliary wheels 33 are rotatably attached to the inside of the device main body 2 such that the device main body 2 can be supported at three points together with the drive wheel 32. The auxiliary wheel 33 is a component that may be added as needed, and is not a necessary component.

By driving the drive wheel 32 by the motor 31, the device main body 2 can be moved along the axis direction of the body support 10 together with the drive wheel 32. Then, when the device main body 2 moves along the axial direction of the body support 10, the gripped portion of the body support 10 can be wiped and cleaned by the wiping member 6 impregnated with the disinfectant solution.

The device drive buttons 4A and 4B and the lighting lamp 5 are provided on the outer peripheral side of the device main body 2. The device drive buttons 4A and 4B are buttons for driving the motor 31, that is, buttons for automatically moving the device main body 2 along the axis direction of the body support 10. In the present embodiment, the lighting lamp 5 lights up when the device drive button 4 is pressed and the device main body 2 automatically moves along the axial direction of the body support 10, that is, when the disinfection device 1 is wiping the gripped portion of the body support 10. The lighting lamp 5 is also a component that may be added as needed, like the auxiliary wheel 33, and is not a necessary component.

In the present embodiment, the device main body 2 is configured to be able to automatically move along the axial direction of the body support 10, but it does not necessarily have to be automatic. For example, a configuration may be configured in which the motor 31 is not provided and the device main body 2 can be manually moved along the axial direction of the body support 10.

According to the present embodiment, the disinfection device 1 attached to the body support 10 gripped by a hand and disinfecting the body support 10 includes the device main body 2 (main body portion) that moves along the body support 10 while in contact with the body support 10. The device main body 2 has the wiping member 6 capable of being impregnated with a disinfectant solution at a part where the wiping member contacts with the body support 10.

Therefore, by moving the device main body 2 along the axial direction of the body support 10, the gripped portion of the body support 10 can be wiped and cleaned by the wiping member 6 impregnated with the disinfectant solution, in such a manner that the gripped portion of the body support 10 can be disinfected. In particular, since the device main body 2 according to the present embodiment is configured to automatically move along the body support 10, the gripped portion of the body support 10 can be wiped and cleaned, and the gripped portion of the body support 10 can be automatically disinfected.

Further, the device main body 2 according to the present embodiment has a storage portion 22 which stores the disinfectant solution thereinside, and also has the communication hole 23 which allows the wiping member 6 to be impregnated with the disinfectant solution stored in the storage portion 22 by communicating the storage portion 22 and the wiping member 6.

As a result, since it is not necessary to spray the disinfectant solution on the holding portion of the body support 10, there is no possibility that the sprayed disinfectant solution adheres to the skin or that the sprayed disinfectant solution is inhaled. Therefore, the effect of the disinfectant solution on the skin and respiratory organs can be reduced.

Further, the device main body 2 according to the present embodiment has a hollow cylindrical shape. The device main body 2 is inserted through the body support 10 such that its inner peripheral surface is in contact with the body support 10, and is attached to the body support 10.

As a result, since the wiping member 6 impregnated with the disinfectant solution can be placed on the inner peripheral surface of the device main body 2, where the wiping member contacts with the body support 10, the wiping member 6 can be covered and hidden by the device main body 2. Therefore, it is possible to prevent the wiping member 6 and, by extension, the disinfectant solution from being directly touched and affecting the skin.

### Second Embodiment

Next, a second embodiment of the present disclosure will be described. The present embodiment is different from the first embodiment in that the operation of the disinfection device 1 is controlled according to the surrounding situations of the disinfection device 1. The differences will be described below.

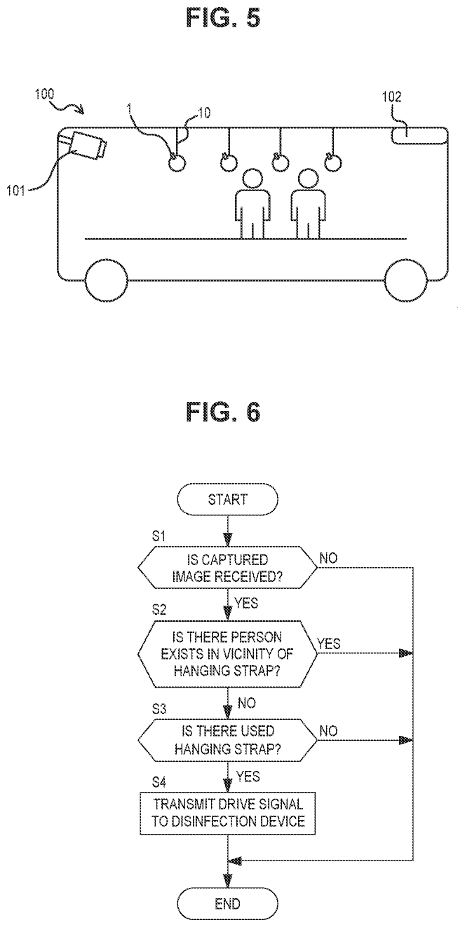

FIG. 5 is a schematic configuration diagram of a disinfection system 100 according to the second embodiment of the present disclosure, which is formed in a bus.

The disinfection system 100 according to the present embodiment includes the disinfection device 1, a recognition sensor 101 that recognizes the surrounding situation of each disinfection device 1, and a control device 102 that controls the operation of each disinfection device 1 in the bus. FIG. 5 illustrates an example in which the disinfection device 1 is attached to the hanging strap as the body support 10. However, the attachment location of the disinfection device 1 is not limited thereto, and may be attached to, for example, a handrail in the bus.

As described above, the recognition sensor 101 is a sensor that recognizes the situation around the disinfection device 1, and in FIG. 5, a camera is shown as an example thereof. The camera as the recognition sensor 101 takes image of the inside of the bus and transmits the captured image to the control device 102.

The control device 102 is, for example, a general-purpose computer having a communication function, and controls the operation of each disinfection device 1 attached to the hanging strap in the bus according to the surrounding situations of the disinfection device 1. In the present embodiment, the control device 102 determines whether a person exists in a vicinity of each disinfection device 1 attached to the hanging strap by performing image processing on the captured image received from the camera which serves as the recognition sensor 101. Then, the control device 102 controls (via remote control) the operation of each disinfection device 1 attached to the hanging strap in the bus based on the determination result. The control device 102 does not necessarily have to be installed inside the bus, and may be installed outside the bus.

FIG. 6 is a flowchart illustrating an example of a process executed by the control device 102 to control the operation of each disinfection device 1 attached to the hanging strap in the bus.

In step S1, the control device 102 determines whether the captured image is received from the recognition sensor 101 (camera). The recognition sensor 101 (camera) periodically takes image of the inside of the bus and transmits the captured image to the control device 102. When the control device 102 receives the captured image, the control device 102 proceeds to the process of step S2. On the other hand, when the control device 102 has not received the captured image, the control device 102 ends the process.

In step S2, the control device 102 performs image processing on the captured image to determine whether there is a person exists in a vicinity of each hanging strap (body support 10) to which the disinfection device 1 is attached. The control device 102 ends the process when there are persons exist in the vicinity of all the hanging straps. On the other hand, the control device 102 proceeds to the process of step S3 when there is a hanging strap where no person is present within its vicinity.

In step S3, the control device 102 determines whether, in the hanging straps where no person is present within its vicinity, there is a hanging strap (hereinafter, referred to as “used hanging strap”) that has been confirmed to have been used by a person when performing image processing on the captured image received during the most recent fixed period (for example, 15 minutes) in the past. The control device 102 proceeds to the process of step S4 when there is a used hanging strap among the hanging straps where no person is present within its vicinity. On the other hand, the control device 102 ends the current process when there is no used hanging strap among the hanging straps where no person is present within its vicinity.

In step S4, the control device 102 transmits a drive signal to the disinfection device 1 attached to the used hanging strap among the hanging straps where no person is present within its vicinity, and drives the disinfection device 1 to disinfect the used hanging strap. In the present embodiment, the motor driver of the motor 31 of each disinfection device 1 is configured to enable wireless communication with the control device 102. In addition, each disinfection device 1 is automatically driven when a drive signal from the control device 102 is received, and can automatically disinfect the body support 10.

The disinfection system 100 according to the present embodiment includes the disinfection device 1 that is attached to the body support 10 that is gripped by a hand, and automatically disinfects the body support 10, the recognition sensor 101, which recognizes the surrounding situations of the disinfection device 1, and the control device 102 that remotely controls the disinfection device 1 based on the surrounding situations of the disinfection device 1 recognized by the recognition sensor 101.

As a result, the body support 10 can be automatically disinfected by driving the disinfection device 1 at the appropriate time according to the surrounding situations of the disinfection device 1.

Especially in the present embodiment, the recognition sensor 101 is configured to detect whether there is a person exists in a vicinity of the body support 10, and the control device 102 is configured to activate the disinfection device 1 to disinfect the body support 10 when there is no person exists in the vicinity of the body support 10.

Therefore, it is possible to prevent people exists in the vicinity of the body support 10 from being inconvenienced during disinfection by the disinfection device 1.

In the present embodiment, a camera is described as an example of the recognition sensor 101, but the recognition sensor 101 is not limited thereto. For example, a touch sensor built in the body support 10 may recognize the situations around the disinfection device 1, that is, whether the body support 10 to which the disinfection device 1 is attached is gripped by a person. That is, the recognition sensor 101 is configured to detect whether the body support 10 is gripped by a hand, and the control device 102 may be configured to activate the disinfection device 1 to disinfect the body support 10 when the body support 10 is not gripped by a hand.

Although the embodiments of the present disclosure are described above, the embodiments show only a part of application examples of the present disclosure, and do not intend to limit the technical scope of the present disclosure to the specific configurations of the embodiments.

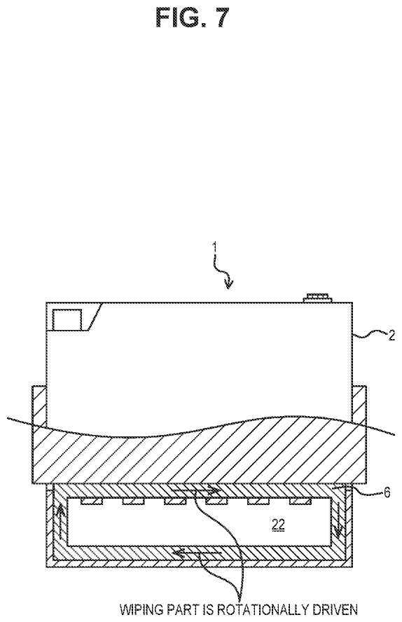

For example, as a modification example of the disinfection device 1 described in the first embodiment, as illustrated in FIG. 7, the wiping member 6 may be configured to be rotatable inside the device main body 2. As a result, it is possible to rotate and move a dirty portion of the handrail once in contact with the gripped portion of the body support 10 and soak it in the disinfectant solution in the device main body 2 so as to clean it. Accordingly, the wiping member 6 can be kept cleaner.

What is claimed is:

1. A disinfection device which is attached to a body support that is gripped by a hand, and disinfects the body support, the disinfection device comprising:
a main body portion configured to move along the body support while in contact with the body support,
wherein the main body portion includes a wiping member configured to be impregnated with a disinfectant solution at a part where the wiping member contacts with the body support.

2. The disinfection device according to claim 1, wherein the main body portion includes a storage portion that stores the disinfectant solution inside and a communication hole that allows the wiping member to be impregnated with the disinfectant solution stored in the storage portion by communicating the storage portion and the wiping member.

3. The disinfection device according to claim 1, wherein the main body portion has a hollow cylindrical shape, and is attached to the body support such that an inner peripheral surface of the main body portion is inserted through the body support to be in contact with the body support.

4. The disinfection device according to claim 1, wherein the main body portion is configured to move automatically along the body support.

5. A disinfection system comprising:
a disinfection device which is attached to a body support that is gripped by a hand, and disinfects the body support;
a sensor configured to recognize surrounding situations of the disinfection device; and
a control device configured to remotely control the disinfection device based on the surrounding situations of the disinfection device recognized by the sensor.

6. The disinfection system according to claim 5, wherein:
the sensor is configured to detect whether the body support is gripped by a hand; and
the control device is configured to, when the body support is not gripped by a hand, activate the disinfection device to disinfect the body support.

7. The disinfection system according to claim 5, wherein:
the sensor is configured to detect whether there is a person who exists in a vicinity of the body support; and
the control device is configured to, when there is no person who exists in the vicinity of the body support, activate the disinfection device to disinfect the body support.
